(19) United States
(12) Patent Application Publication
(10) Pub. No.: US 2026/0325038 A1
(43) Pub. Date: Oct. 22, 2026

(54) DEVICE AND METHOD FOR GENERATING ELECTROCARDIOGRAM RECONSTRUCTION MODEL, AND METHOD FOR RECONSTRUCTING ELECTROCARDIOGRAM

(71) Applicants: KNU-INDUSTRY COOPERATION FOUNDATION, Chuncheon-Si (KR); EWHA UNIVERSITY - INDUSTRY COLLABORATION FOUNDATION, Seoul (KR)

(72) Inventors: Hyeon Seung Im, Chuncheon-Si (KR); Jun Beom Park, Seoul (KR); Gi Hun Joo, Chuncheon-Si (KR); Jin Ho Joo, Goyang-Si (KR)

(73) Assignees: KNU-INDUSTRY COOPERATION FOUNDATION, Chuncheon-Si (KR); EWHA UNIVERSITY - INDUSTRY COLLABORATION FOUNDATION, Seoul (KR)

(21) Appl. No.: 18/866,218

(22) PCT Filed: May 10, 2024
(86) PCT No.: PCT/KR2024/006374

§ 371 Date: Nov. 15, 2024

(30) Foreign Application Priority Data
Nov. 14, 2023 (KR) 10-2023-0157205

(51) Int. Cl. A61B 5/327; A61B 5/308; A61B 5/346

(52) U.S. Cl. CPC A61B 5/327; A61B 5/308; A61B 5/346

(57) ABSTRACT

The disclosure relates to a device for generating an electrocardiogram (ECG) reconstruction model based on a Generative Adversarial Network (GAN) including an input unit configured to receive a single-lead electrocardiogram as input, a generator configured to reconstruct a 12-lead electrocardiogram based on the single-lead electrocardiogram, and a discriminator configured to determine whether the input is a reconstructed 12-lead electrocardiogram or a true 12-lead electrocardiogram using either a first input of said 12-lead electrocardiogram plus the single-lead electrocardiogram or a second input of a ground-truth plus the single-lead electrocardiogram as an input value, the discriminator determining whether the input value is a reconstructed 12-lead electrocardiogram or a true 12-lead electrocardiogram. A multi-lead electrocardiogram that more faithfully reflects the characteristics of the actual electrocardiogram can be reconstructed from a single-lead electrocardiogram.

### TECHNICAL FIELD

The disclosed invention relates to a device and method for generating an electrocardiogram (ECG) reconstruction model and a method for reconstructing electrocardiograms based on generative adversarial networks, which can reconstruct multiple-lead electrocardiograms from single-lead electrocardiograms.

The present invention was partly supported by “Regional Innovation Strategy (RIS)” through the National Research Foundation of Korea (NRF) funded by the Ministry of Education (MOE) (2022RIS-005, 50%) and Institute of Information & Communications Technology Planning & Evaluation (IITP) grant funded by the Korea government (MSIT) ((No. RS-2022-00155966, Artificial Intelligence Convergence Innovation Human Resources Development (Ewha Womans University), 30%) and (IITP-2024-RS-2023-00260267, Innovative Human Resource Development for Local Intellectualization program, 20%)).

### BACKGROUND ART

An electrocardiogram (ECG) is a graphic representation of the electrical activity of the heart that occurs during a heartbeat cycle. An ECG test is an essential test for diagnosing heart abnormalities and diagnosing heart arrhythmia with irregular heart rates, and detects the electrical activity of the heart by attaching electrodes to the skin. A representative ECG test method is the standard 12-lead ECG test, which uses six limb leads (Lead I, II, III, aVR, aVL, aVF) where electrode cables are attached to the extremities, and six chest leads (V1, V2, V3, V4, V5, V6) where electrode cables are attached to fixed locations on the chest.

FIG. 1 shows a typical ECG image. As shown in FIG. 1, each lead of the ECG looks similar, but contains different information about the heart. Generally, in order to diagnose heart disease, different leads need to be observed for each disease. Information about the heart that can be obtained from a single lead is inevitably limited. Therefore, when diagnosing heart disease through an ECG, various leads must be observed organically and a comprehensive judgment must be made.

Recently, ECG monitoring has become possible through smartwatches, such as Galaxy Watch and Apple Watch, and smart devices, such as portable ECG monitors. However, most of these smart devices can only measure single-lead ECGs. In this case, simple atrial fibrillation can be diagnosed with just a single lead, but other heart diseases are difficult to measure, which is a disadvantage.

In recent years, studies on improving the accuracy of diagnosing heart disease and predicting related diseases using ECGs have been appearing one after another. The method of measuring ECGs using smart devices has the advantages of being relatively easy and convenient to measure and being able to record for a long time, but as mentioned above, most ECGs measured using smart devices are single-lead ECGs. Therefore, there is a disadvantage in that diagnostic technologies using 12-lead ECGs that have been actively studied so far cannot be applied.

In the case of conventional technologies, there are methods that reconstruct missing ECGs using an artificial intelligence model based on a short-term and long-term memory neural network, as disclosed in Korean Patent No. 10-2570598 (publication date: Aug. 28, 2023), or generate the remaining 12-n ECGs from information of n electrodes based on an adversarial neural network algorithm, as disclosed in Korean Patent No. 10-2412974 (publication date: Jun. 24, 2022). However, these conventional technologies simply aim to generate or reconstruct ECGs well, and do not perform detailed comparisons of pixels or time units. In addition, in order to diagnose diseases using ECGs, it is necessary to confirm whether the reconstructed ECG reflects the characteristics of the actual ECG well. In other words, a technology is required that allows the reconstructed ECG to reflect the characteristics of the existing actual ECG well, rather than simply reconstructing the ECG.

### DISCLOSURE

### Technical Problem

For the above reasons, one aspect of the disclosed invention is to provide a device for generating electrocardiogram reconstruction model, a method for generating electrocardiogram reconstruction model, and a method for reconstructing electrocardiograms based on generative adversarial networks capable of reconstructing multiple electrocardiograms from a single electrocardiogram so as to accurately reflect the characteristics of an actual electrocardiogram.

### Solution to Problem

In order to achieve this purpose, in one general aspect, the present invention provides a device for generating an electrocardiogram (ECG) reconstruction model, generating an ECG reconstruction model based on a generative adversarial network (GAN), including an input unit configured to receive an input of a single-lead ECG; a generator configured to reconstruct a 12-lead ECG based on the single-lead ECG; and a discriminator configured to use as an input value either a first input value that is a sum of the 12-lead ECG and the single-lead ECG or a second input value that is a sum of a ground-truth 12-lead ECG and the single-lead ECG and determine whether the input value is the reconstructed 12-lead ECG or the ground-truth 12-lead ECG.

The generator may include: a label generator configured to receive the single-lead ECG as input and output a second latent vector for outputting a single-lead ECG identical to the single-lead ECG based on a U-Net-based encoder-decoder generation model without applying skip connections; and an inference generator configured to perform preprocessing on the single-lead ECG based on a U-Net-based encoder-decoder generation model and reconstruct and output the 12-lead ECG based on a first latent vector and the second latent vector of the preprocessed single-lead ECG.

The inference generator may perform learning based on an adversarial loss function for the first input value and the second input value.

The inference generator may perform learning based on an L1 loss function that reflects the Manhattan Distance between the 12-lead ECG and the ground-truth 12-lead ECG.

The inference generator may perform learning based on a latent vector loss function for the first latent vector and the second latent vector.

The inference generator may perform learning based on a total loss function that gives respective weights to the adversarial loss function, the L1 loss function, and the latent vector loss function.

The discriminator may be a 1D U-Net-based encoder-decoder generation model.

In another general aspect, the present invention provides a method for generating an electrocardiogram (ECG) reconstruction model, based on a generative adversarial network (GAN), including: receiving as input a single-lead ECG;

reconstructing 12-lead ECG based on the single-lead ECG; receiving as an input value either a first input value that is a sum of the 12-lead ECG and the single-lead ECG or a second input value that is a sum of a ground-truth 12-lead ECG and the single-lead ECG; and determining whether the input value is the reconstructed 12-lead ECG or the ground-truth 12-lead ECG.

The reconstruction of 12-lead ECG based on the single-lead ECG may include: performing preprocessing on the single-lead ECG based on a U-Net-based encoder-decoder generation model; receiving the single-lead ECG as input and outputting a second latent vector for outputting a single-lead ECG identical to the single-lead ECG based on a U-Net-based encoder-decoder generation model without applying skip connections; and reconstructing and outputting the 12-lead ECG based on a first latent vector and the second latent vector of the single-lead ECG preprocessed.

The method for generating an electrocardiogram (ECG) reconstruction model may include performing learning based on an adversarial loss function for the first input value and the second input value.

The method for generating an electrocardiogram (ECG) reconstruction model may include performing learning based on an L1 loss function reflecting the Manhattan Distance between the 12-lead ECG and the ground-truth 12-lead ECG.

The method for generating an electrocardiogram (ECG) reconstruction model may include performing learning based on a latent vector loss function for the first latent vector and the second latent vector.

The method for generating an electrocardiogram (ECG) reconstruction model may include performing learning based on a total loss function in which weights are respectively assigned to the adversarial loss function, the L1 loss function, and the latent vector loss function.

In another general aspect, the present invention provides a method for reconstructing electrocardiograms (ECGs), using an ECG reconstruction model based on a generative adversarial network, including: receiving a single-lead ECG as input; preprocessing the single-lead ECG; inputting the single-lead ECG into an ECG reconstruction model based on a pre-trained generative adversarial network to reconstruct a 12-lead ECG; and outputting the 12-lead ECG reconstructed.

### Effects of Invention

According to one aspect of the disclosed invention, there can be provided a device for generating an electrocardiogram (ECG) reconstruction model, a method for generating an electrocardiogram reconstruction model, and a method for reconstructing electrocardiograms based on generative adversarial networks capable of reconstructing multiple-lead electrocardiograms which more accurately reflects the characteristics of an actual electrocardiogram from single-lead electrocardiograms.

According to one aspect of the disclosed invention, reconstructing a multi-lead electrocardiogram from only a single-lead electrocardiogram can save medical professionals money and time in diagnosing heart disease.

According to one aspect of the disclosed invention, in addition to an electrocardiogram, when specific multiple signals exist, a missing signal can be reconstructed or a signal that is difficult to measure can be reconstructed from another signal, so that it can be applied to various fields such as various brain wave or cerebral blood flow measurements.

### BRIEF DESCRIPTION OF DRAWINGS

FIG. 1 is a graph showing an image of typical 12-lead electrocardiogram (ECG).

FIG. 2 is a drawing schematically showing the configuration of a device for generating an ECG reconstruction model 100 according to an embodiment of the present invention.

FIG. 3 schematically illustrates an ECG reconstruction model (EKGAN) according to an embodiment of the present invention.

FIG. 4 is a flow chart showing a method for generating an ECG reconstruction model according to an embodiment of the present invention.

FIG. 5 is a flow chart showing more specifically the step for reconstructing a 12-lead ECG in the method for generating an ECG reconstruction model according to an embodiment of the present invention.

FIG. 6 is a diagram comparing the reconstruction performance of an ECG reconstruction model (EKGAN) according to an embodiment of the present invention and existing models.

FIG. 7 is a diagram showing the performance difference according to the presence or absence of a discriminator and a label generator in an ECG reconstruction model (EKGAN) according to an embodiment of the present invention.

FIG. 8 is a diagram comparing the prediction performance for three diseases of a 12-lead ECG reconstructed by an ECG reconstruction model (EKGAN) according to an embodiment of the present invention, a 12-lead ECG reconstructed by an existing model, and an actual 12-lead ECG.

FIG. 9 is a diagram comparing the reconstruction performance of the ECG reconstruction model (EKGAN) according to an embodiment of the present invention and existing models.

### BEST MODE FOR IMPLEMENTATION OF THE INVENTION

Throughout the specification, the same reference numerals refer to the same components. This specification does not describe all elements of the embodiments, and any content that is general in the technical field to which the disclosed invention belongs or that overlaps between the embodiments is omitted. The terms ‘unit, part, module, member, element, block’ used in the specification may be implemented in software or hardware, and depending on the embodiments, multiple ‘units, parts, modules, members, elements, blocks’ may be implemented as one component, or one ‘unit, part, module, member, element, block’ may include multiple components.

Throughout the specification, when a part is said to be ‘connected’ to another part, this includes not only direct connection but also indirect connection, and indirect connection includes connection via a wireless communications network.

Additionally, when a part is said to ‘comprise, include’ a component, this does not mean that it excludes other components, unless otherwise specifically stated, but rather that it may include other components.

Throughout the specification, when we say that an element is located ‘on’ another element, this includes not only cases where the element is in contact with the other element, but also cases where there is another element between the two elements.

The terms first, second, etc. are used to distinguish one component from another, and the components are not limited by the aforementioned terms.

Singular expressions include plural expressions unless the context clearly indicates otherwise.

The identification codes in each step are used for convenience of explanation and do not describe the order of each step. Each step may be performed in a different order than specified unless the context clearly indicates a specific order.

The working principle and embodiments of the disclosed invention will be described with reference to the attached drawings below.

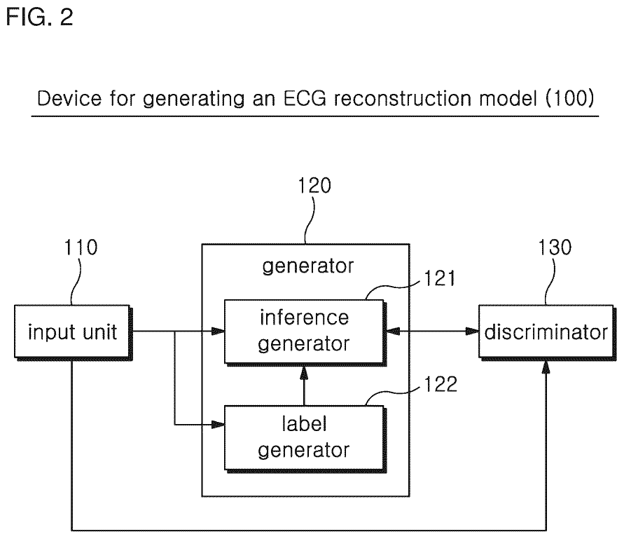

FIG. 2 is a drawing schematically showing the configuration of a device for generating an electrocardiogram (ECG) reconstruction model 100 according to an embodiment.

Referring to FIG. 2, the device for generating an ECG reconstruction model 100 according to an embodiment of the present invention may include an input unit 110, a generator 120, and a discriminator 130.

The input unit 110 may receive a single-lead ECG of a specific patient. The input unit 110 may preprocess the single-lead ECG. In addition, the input unit 110 may provide the single-lead ECG or the preprocessed single-lead ECG to the generator 120.

According to an embodiment, the input unit 110 may additionally receive an actual 12-lead ECG (ground-truth) of the specific patient. The input unit 110 may provide the actual 12-lead ECG to the discriminator 130. The generator 120 may reconstruct a 12-lead ECG based on a single-lead ECG provided from the input unit 110.

The generator 120 may reconstruct a 12-lead ECG based on a single-lead ECG provided from the input unit 110.

To this end, the generator 120 may include an inference generator 121 and a label generator 122.

The inference generator 121 performs preprocessing on a single-lead ECG based on a U-Net-based encoder-decoder generation model, and may reconstruct and output a 12-lead ECG based on a first latent vector of the preprocessed single-lead ECG and a second latent vector provided from the label generator 122 to be described later.

The label generator 122 may receive the single-lead ECG based on a U-Net-based encoder-decoder generation model that does not apply skip connections and output a second latent vector for outputting the same single-lead ECG as the single-lead ECG.

The discriminator 130 may use either a first input value that is a combination of a reconstructed 12-lead ECG and a single-lead ECG or a second input value that is a combination of an actual 12-lead ECG and a single-lead ECG as an input value to determine whether the input value is a reconstructed 12-lead ECG or an actual 12-lead ECG.

According to an embodiment, the discriminator 130 may be a 1D U-Net based encoder-decoder generation model.

According to an embodiment, the inference generator 121 may perform learning based on an adversarial loss function for the first input value and the second input value.

According to an embodiment, the inference generator 121 may perform learning based on an L1 loss function that reflects the Manhattan Distance between the reconstructed 12-lead ECG and the actual 12-lead ECG.

According to an embodiment, the inference generator 121 may perform learning based on a latent vector loss function for the first latent vector and the second latent vector.

According to an embodiment, the inference generator 121 may perform learning based on a total loss function that assigns respective weights to the adversarial loss function, the L1 loss function, and the latent vector loss function.

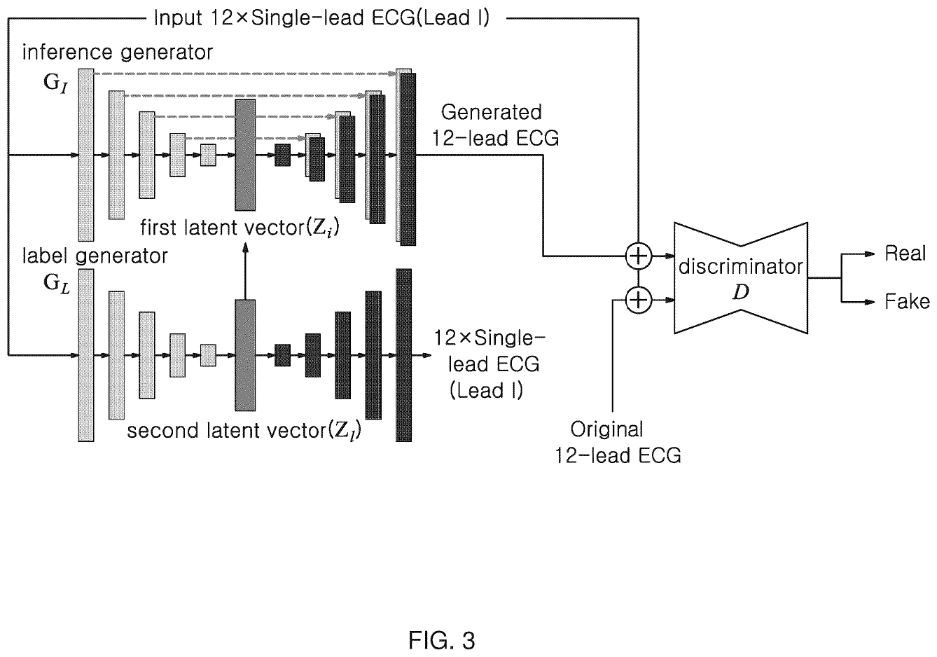

FIG. 3 schematically illustrates an ECG reconstruction model (EKGAN) according to one embodiment.

The process of reconstructing a single-lead ECG to a multi-lead ECG may be performed based on a generative adversarial network (GAN). However, in the case of a general GAN model, there may be a problem in that it does not reflect the characteristics of an actual (diseased) ECG well. In order to overcome the limitations of reconstructing a single-lead ECG to a multi-lead ECG using an existing GAN model, an ECG reconstruction model (EKGAN) according to the present invention is disclosed.

Referring to FIG. 3, the ECG reconstruction model (EKGAN) may include a generator 120 including an inference generator Gₗ, 121, a label generator G_{L}, 122, and a discriminator D, 130. In addition to this, the ECG reconstruction model (EKGAN) may further include a preprocessing unit (not shown).

The generator 120 plays a role in reconstructing a 12-lead ECG when a single-lead ECG is input. The generator 120 includes two sub-generators, namely, an inference generator Gₗ, 121, and a label generator G_{L}, 122, each of which performs a different role and may organically proceed with learning.

The inference generator Gₗ, 121, is based on the U-Net-based encoder-decoder generation model to receive a single-lead ECG signal as input and reconstruct it to a 12-lead ECG. At this time, preprocessing of the input signal may be performed at the input terminal of the encoder to configure the encoder-decoder-based model. Afterwards, when the output generated at the output terminal is transferred to the input of the discriminator, the discriminator may discriminate between real and fake and use this to proceed with learning.

The detailed structure of the inference generator (Gₗ) 121 may include an input terminal, a U-Net-based encoder-decoder generation model, and an output terminal.

The input terminal receives a single-lead ECG as input. Here, the single-lead ECG has a size of (1, n, 1). Here, the first 1 refers to the number of leads, n refers to the length of the ECG, and the last 1 refers to the channel size. Here, the length n can be set in various ways by the user, and in the present invention, the value n=512 is used for explanation.

In order to use the encoder-decoder based model, the input and output sizes must be the same, and a preprocessing step is required for the single-lead ECG entering the input terminal. To do this, the single-lead ECG is simply copied 12 times and zero-padded at the front and back to change the size to (16, 512, 1). At this time, each convolution filter is configured in the order of 64, 128, 256, 512, and 1024.

The U-Net-based encoder-decoder generation model may include a Contracting Path, which extracts semantic information by viewing a gradually wider range of image pixels as a part corresponding to the encoder, a Bottleneck, which generates a first latent vector Zi as a section transitioning from the contracting path to the expanding path, and an Expanding Path, which combines the extracted semantic information using pixel location information and skip connections as a part corresponding to the decoder and provides additional information to each pixel.

U-Net is a model that can extract image features using information that can be identified in high dimensions as well as low dimensions, and combines this with location information to achieve good performance.

U-Net is based on providing additional information to the decoder that could be lost when compressing input data into latent vectors.

However, the latent vector generated from the existing U-Net model has a disadvantage in that it does not check how well the latent vector includes the features of the input data because it is updated only using the loss function between the original data and the generated data. To solve this problem, the present invention uses a technique that helps compress the input data without losing information by introducing a label generator G_{L}, 122. A detailed description of this will be described later in the description of the label generator G_{L}, 122.

The output terminal is configured to have an output of size (16, 512, 1) such that the output is the same size as the input of size (16, 512, 1) preprocessed at the input terminal. The output terminal uses the first latent vector Zi of the single-lead ECG data passed through the input terminal to reconstruct it to a 12-lead ECG. Since it is a U-Net-based structure, it learns to make the best use of the information in the single-lead ECG through skip connection. The ECG restored at the output terminal is configured in the order of Lead I, II, III, aVR, aVL, aVF, V1-V6, with zero padding, and each convolution filter may be configured in the order of 512, 256, 128, 64, 1.

The label generator G_{L}, 122, is used to minimize information loss during compression of the input data based on U-Net. To this end, the label generator G_{L}, 122, is configured as an encoder-decoder-based model to receive the same single-lead ECG as input from the inference generator Gₗ, 121, and generate the same single-lead ECG output. The key role of the label generator G_{L}, 122, is to find the second latent vector Z_{I} that can best represent the single-lead ECG received as input, and to this end, it induces the generation of an ECG identical to the input. In other words, since the second latent vector Z_{I} alone can generate a single-lead ECG well, this means that the second latent vector Z_{I} can be compressed while retaining much of the information of the single-lead ECG received as input.

The overall structure of the label generator G_{L}, 122, is the same as that of the inference generator Gₗ, 121, but skip connection is not applied to compress information as much as possible. This differentiates the label generator G_{L}, 122, from a typical U-Net-based encoder-decoder generation model, wherein the second latent vector Zₗ generated by the label generator G_{L}, 122, can improve the information compression capability of the inference generator Gₗ, 121, through comparison with the first latent vector Zᵢ generated from the input data of the inference generator Gₗ, 121, and the loss function between them.

Each lead of a 12-lead ECG has unique characteristics. Therefore, it is very important for the discriminator D 130 to individually analyze each lead signal at the pixel level.

Therefore, when using a 2D convolution-based discriminator used in GAN models for general image generation, the unique characteristics of each lead may be intermixed with those of other leads, which may degrade the reconstruction quality.

To prevent this problem, the discriminator may use a 1D U-Net-based encoder-decoder generative model. The discriminator uses the reconstructed 12-lead ECG that is the output of the inference generator Gₗ, 121 or the actual 12-lead ECG, as an input by combining it with the single-read ECG used as an input, and learns to determine whether the input value is a generated ECG or an original ECG.

In other words, the discriminator uses as input the first input value which is the sum of the reconstructed 12-lead ECG output from the inference generator Gₗ, 121, and the single-lead ECG used as input, or the second input value which is the sum of the actual 12-lead electrocardiogram and the single-lead electrocardiogram used as input.

### Mode for Implementation of the Invention

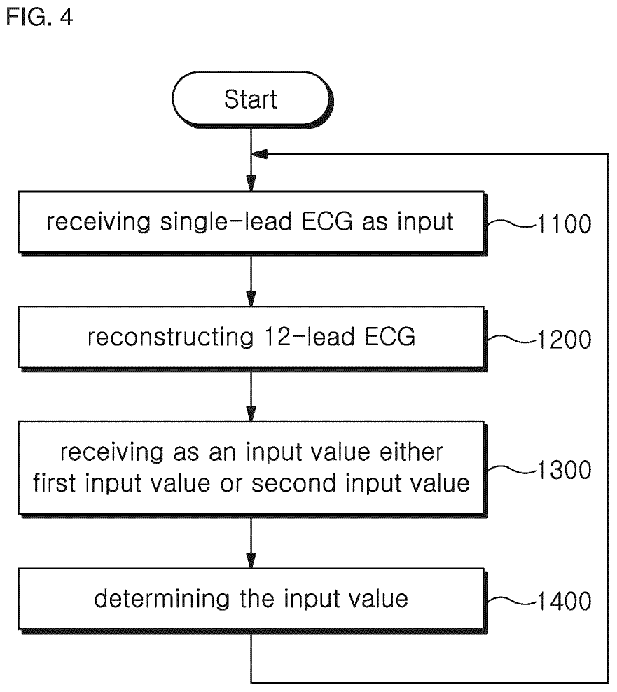

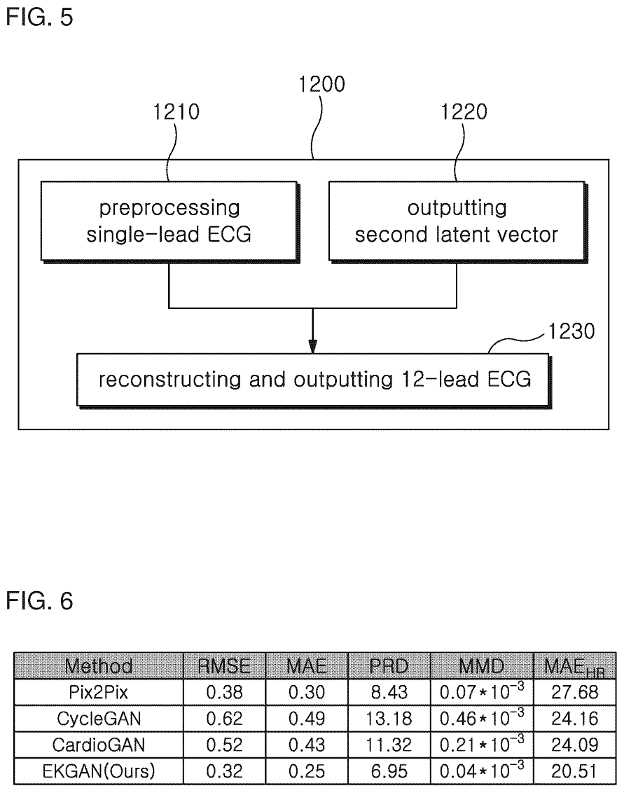

FIG. 4 is a flow chart showing a method for generating an ECG reconstruction model according to an embodiment. FIG. 5 is a flow chart showing more specifically the steps for reconstructing a 12-lead ECG in the method for generating an ECG reconstruction model according to an embodiment.

Referring to FIG. 4, a method for generating an ECG reconstruction model according to an embodiment of the present invention may include: receiving a single-lead ECG as input (1100); reconstructing a 12-lead ECG based on the single-lead ECG (1200); receiving as an input value either a first input value that is a sum of the 12-lead ECG and the single-lead ECG or a second input value that is a sum of an actual 12-lead ECG (ground-truth) and the single-lead ECG (1300); and determining whether the input value is a reconstructed 12-lead ECG or an actual 12-lead ECG (1400).

Referring to FIG. 5, a step (1200) of reconstructing a 12-lead ECG based on a single-lead ECG may include: performing preprocessing on the single-lead ECG based on a U-Net-based encoder-decoder generation model (1210); receiving the single-lead ECG based on a U-Net-based encoder-decoder generation model that does not apply skip connections and outputting a second latent vector for outputting a single-lead ECG identical to the above single-lead ECG (1220); and reconstructing and outputting the 12-lead ECG based on the first latent vector of the preprocessed single-lead ECG and the second latent vector (1230).

According to an embodiment, the method for generating an ECG reconstruction model may further include a step (not shown) of performing learning based on an adversarial loss function for the first input value and the second input value.

According to an embodiment, the method for generating an ECG reconstruction model may further include a step (not shown) of performing learning based on an L1 loss function reflecting the Manhattan Distance between the reconstructed 12-lead ECG and the actual 12-lead ECG.

According to an embodiment, the method for generating an ECG reconstruction model may further include a step (not shown) of performing learning based on a latent vector loss function for the first latent vector and the second latent vector.

According to an embodiment, the method for generating an ECG reconstruction model may further include a step (not shown) of performing learning based on a total loss function in which respective weights are applied to the adversarial loss function, the L1 loss function, and the latent vector loss function.

An ECG reconstruction method (not shown) according to an embodiment of the present invention may include receiving a single-lead ECG, preprocessing the single-lead ECG, reconstructing a 12-lead electrocardiogram by inputting the single-lead ECG into an ECG reconstruction model based on a pre-trained generative adversarial network, and outputting the reconstructed 12-lead ECG.

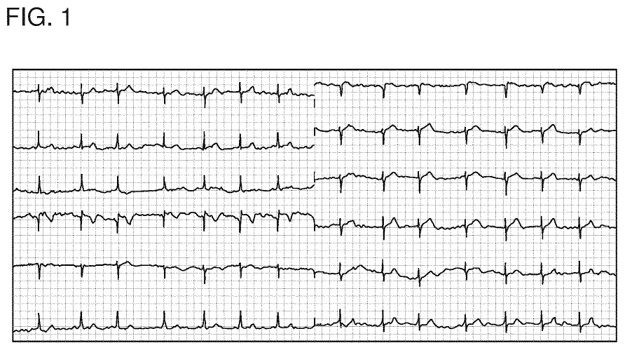

The ECG reconstruction method according to an embodiment assumes the use of a pre-learned ECG reconstruction model (EKGAN) disclosed in FIGS. 1 to 3. Therefore, a 12-lead ECG can be reconstructed based on a single-lead ECG inputted using an ECG reconstruction model (EKGAN) that already has high performance.

Meanwhile, in the present invention, the generator 120 and the discriminator 130 may use a loss function for learning the ECG reconstruction model (EKGAN). The loss functions used at this time are largely divided into three types, including an adversarial loss function, an L1 loss function, and a latent vector loss function, and a total loss function that assigns weights to each of these three functions may be additionally used.

Before explaining each loss function, the meaning of the symbols used in each loss function is as follows.
Gₗ: inference generator 121
G_{L}: label generator 122
D: discriminator 130
eᵢ: input data of single-lead ECG copied 12 times and zero-padded
eₒ: the corresponding ground-truth 12-lead ECG for a single-lead ECG
zᵢ, Z_{I}: latent vectors produced by Gₗ and G_{L}, respectively.

The adversarial loss function is used as a basis for learning about the difference between the data reconstructed from the single-lead ECG received as input by the inference generator Gₗ, 121, and the ground-truth 12-lead ECG. The equation is as follows: it is a Conditional GAN-type loss function on the result of inputting the actual data into the discriminator D, 130, and the reconstructed 12-lead ECG data obtained from inputting the input data into the inference generator Gₗ, 121, and the input data. In other words, the discriminator D, 130 learns whether the input value is generated data or actual data by using the adversarial loss function using the ECG used for reconstruction and the actual ECG, and the ECG used for reconstruction and the reconstructed ECG.

[symbol]

_{adv}(G₁,D)= [symbol] ₑ_{ᵢ}_{,e}_{ₒ}[log D(eᵢ,eₒ)]+ [symbol] ₑ_{ᵢ}[log(1-D(eᵢ,Gₗ(eᵢ)))]

The L1 loss function may additionally use the L1 loss that reflects the Manhattan Distance between the real ECG and the generated ECG, in addition to the adversarial loss. This can be used in the same way in the label generator G_{L}, 122, to help the inference generator Gₗ, 121, in addition to the inference generator Gₗ, 121, that performs the actual reconstruction, and the detailed formula is as follows.

[symbol]

_{L1}(Gᵣ)= [symbol] ₑ_{ᵢ}_{,e}_{ₒ}[∥eₒ−G_{I}(eᵢ)∥₁] [symbol] _{L1}(G_{L})= [symbol] eᵢ[∥eᵢ−G_{L}(eᵢ)∥₁]

The latent vector loss function can calculate the L1 loss between the second latent vector Z_{I} generated from the label generator G_{L}, 122, and the first latent vector Zᵢ generated from the inference generator Gₗ, 121, to check the similarity between the two latent vectors. The detailed formula is as follows.

[symbol]

_{LV}(G_{I},G_{L})= [symbol] _{z}_{ᵢ}_{z}_{ₗ}[∥zᵢ−z₁∥1]

The total loss function is the summation of the above loss functions into the loss of the inference generator Gₗ, 121, and is given by the following formula. λ and α are variables that control relative importance and can be set differently depending on the given purpose. In embodiments of the present invention, λ=50, α=1 are set.

$${G}_{I}^{*} = arg {min}_{{G}_{I} , {G}_{L}} {max}_{D} \{ {ℒ}_{adv} ( {G}_{I} , D ) + {λℒ}_{L 1} ( {G}_{I} ) + {αℒ}_{LV} ( {G}_{I} , {G}_{L} ) \}$$

Referring to FIGS. 6 to 9, the ECG reconstruction model (EKGAN) according to embodiments of the present invention receives Lead I, which is a single-lead ECG, as input and outputs a reconstructed 12-lead ECG. In order to verify the reconstruction performance of the ECG reconstruction model (EKGAN), learning and verification were performed using ECGs collected at Ewha Womans University Mokdong Hospital and Ewha Womans University Seoul Hospital, and a comparison was made with existing GAN-based models. In addition, performance measurements were performed to verify the usefulness of the label generator G_{L}, 122. Root Mean Square Error (RMSE), Mean Absolute Error (MAE), Percentage Root Mean Square Difference (PRD), Maximum Mean Discrepancy (MMD), and Mean Absolute Error for Heart Rates (MAEHR) were used as performance measurement indicators.

FIG. 6 is a diagram comparing the reconstruction performance of an ECG reconstruction model (EKGAN) according to an embodiment and existing models.

As can be seen in FIG. 6, it can be confirmed that it shows better performance in all indicators than the existing well-known GAN models such as Pix2Pix, CycleGAN, and CardioGAN.

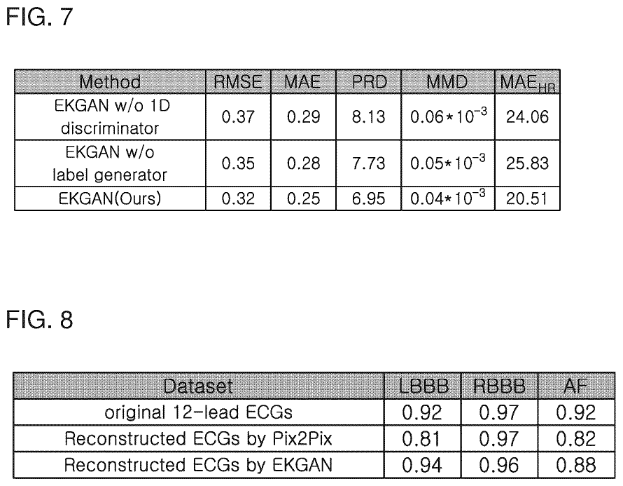

FIG. 7 is a diagram showing the performance difference according to the presence or absence of a discriminator D, 130, and a label generator G_{L}, 122, in an ECG reconstruction model (EKGAN) according to an embodiment.

Referring to FIG. 7, the EKGAN w/o 1D discriminator D, 130, shows a case of using the PatchGAN discriminator used in Pix2Pix rather than using the 1D U-Net model-based discriminator D, 130, in the electrocardiogram reconstruction model (EKGAN) according to an embodiment of the present invention. It can be seen that the performance decreases.

In addition, the EKGAN w/o label generator G_{L}, 122, shows the reconstruction performance when the label generator G_{L}, 122, is not used in the ECG reconstruction model (EKGAN) according to embodiments of the present invention, and it can be confirmed that the performance is lower than that of the ECG reconstruction model (EKGAN) to which the label generator G_{L}, 122, is applied.

FIG. 8 is a diagram comparing the prediction performance for three diseases of a 12-lead ECG reconstructed by an ECG reconstruction model (EKGAN) according to an embodiment, a 12-lead ECG reconstructed by an existing model, and an actual 12-lead ECG.

Referring to FIG. 8, the prediction results of the existing well-known ECG classification model, the prediction results of the original ECG, and the prediction results of the reconstructed ECG are compared, and it can be seen that the disease prediction (classification result) using the ECG reconstruction model (EKGAN) of the present invention shows excellent performance. In this experiment, the prediction performance was confirmed for three diseases that can be diagnosed through ECG. In the case of atrial fibrillation (AF), detection is possible with a single lead, but in the case of Left Bundle Branch Block (LBBB) and Right Bundle Branch Block (RBBB), detection is possible only when various leads are confirmed. In this way, it was experimentally proven through the results of FIG. 8 that the 12-lead ECG reconstructed using the ECG reconstruction model (EKGAN) according to embodiments of the present invention can be applied to various downstream tasks.

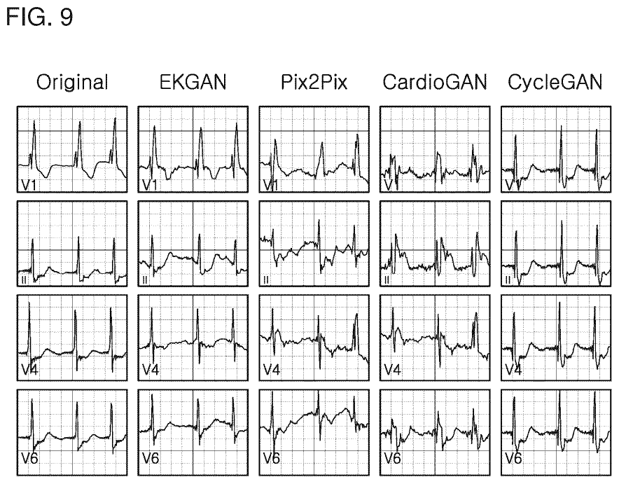

FIG. 9 is a diagram comparing the reconstruction performance of the ECG reconstruction model (EKGAN) according to an embodiment of the present invention and existing models. More specifically, FIG. 9 is a comparison of a 12-lead ECG and a portion of the original ECGs (V1, Lead II, V4, V6) actually reconstructed by the ECG reconstruction model (EKGAN) according to embodiments of the present invention and the models used in the performance comparison.

Referring to FIG. 9, it can be seen that the ECG reconstruction model (EKGAN) according to embodiments of the present invention reconstructs an ECG having a significant similarity to the actual original ECG compared to other models. This indicates that the EKGAN according to embodiments of the present invention can capture characteristics of a disease well from single-lead ECGs and reconstruct 12-lead ECGs relatively accurately compared to other models.

While the above has been described with reference to the illustrated embodiments of the invention, they are exemplary only, and it will be apparent to one having ordinary skill in the art to which the invention belongs that various modifications, changes, and equivalents are possible without departing from the spirit and scope of the invention. The true scope of technical protection of the invention should therefore be determined by the technical ideas of the appended claims.

### INDUSTRIAL APPLICABILITY

The present invention can be used in wearable devices and diagnostic applications, electrocardiogram diagnostic equipment, etc., which enable more accurate and rapid diagnosis using 12-lead electrocardiogram reconstruction techniques.

What is claimed is:

1. A device for generating an electrocardiogram (ECG) reconstruction model, generating an ECG reconstruction model based on a generative adversarial network (GAN), comprising:
an input unit configured to receive an input of a single-lead ECG;
a generator configured to reconstruct a 12-lead ECG based on the single-lead ECG; and
a discriminator configured to use as an input value either a first input value that is a sum of the 12-lead ECG and the single-lead ECG or a second input value that is a sum of a ground-truth 12-lead ECG and the single-lead ECG and determine whether the input value is the reconstructed 12-lead ECG or the ground-truth 12-lead ECG.

2. The device of claim 1,
wherein the generator comprises:
a label generator configured to receive the single-lead ECG as input and output a second latent vector for outputting a single-lead ECG identical to the single-lead ECG based on a U-Net-based encoder-decoder generation model without applying skip connections; and
an inference generator configured to perform preprocessing on the single-lead ECG based on a U-Net-based encoder-decoder generation model and reconstruct and output the 12-lead ECG based on a first latent vector and the second latent vector of the preprocessed single-lead ECG.

3. The device of claim 2,
wherein the inference generator performs learning based on an adversarial loss function for the first input value and the second input value.

4. The device of claim 3,
wherein the inference generator performs learning based on an L1 loss function that reflects the Manhattan Distance between the 12-lead ECG and the ground-truth 12-lead ECG.

5. The device of claim 4,
wherein the inference generator performs learning based on a latent vector loss function for the first latent vector and the second latent vector.

6. The device of claim 5,
wherein the inference generator performs learning based on a total loss function that gives respective weights to the adversarial loss function, the L1 loss function, and the latent vector loss function.

7. The device of claim 1,
wherein the discriminator is a 1D U-Net-based encoder-decoder generation model.

8. A method for generating an electrocardiogram (ECG) reconstruction model, based on a generative adversarial network (GAN), comprising:
receiving as input a single-lead ECG;
reconstructing 12-lead ECG based on the single-lead ECG;
receiving as an input value either a first input value that is a sum of the 12-lead ECG and the single-lead ECG or a second input value that is a sum of a ground-truth 12-lead ECG and the single-lead ECG; and
determining whether the input value is the reconstructed 12-lead ECG or the ground-truth 12-lead ECG.

9. The method of claim 8,
wherein the reconstruction of 12-lead ECG based on the single-lead ECG comprises:
performing preprocessing on the single-lead ECG based on a U-Net-based encoder-decoder generation model;
receiving the single-lead ECG as input and outputting a second latent vector for outputting a single-lead ECG identical to the single-lead ECG based on a U-Net-based encoder-decoder generation model without applying skip connections; and
reconstructing and outputting the 12-lead ECG based on a first latent vector and the second latent vector of the single-lead ECG preprocessed.

10. The method of claim 9, further comprising:
performing learning based on an adversarial loss function for the first input value and the second input value.

11. The method of claim 10, further comprising:
performing learning based on an L1 loss function reflecting the Manhattan Distance between the 12-lead ECG and the ground-truth 12-lead ECG.

12. The method of claim 11, further comprising:
performing learning based on a latent vector loss function for the first latent vector and the second latent vector.

13. The method of claim 12, further comprising:
performing learning based on a total loss function in which weights are respectively assigned to the adversarial loss function, the L1 loss function, and the latent vector loss function.

14. A method for reconstructing electrocardiograms (ECGs), using an ECG reconstruction model based on a generative adversarial network, comprising:
receiving a single-lead ECG as input;
preprocessing the single-lead ECG;
inputting the single-lead ECG into an ECG reconstruction model based on a pre-trained generative adversarial network to reconstruct a 12-lead ECG; and
outputting the 12-lead ECG reconstructed.
